(19) United States
(12) Patent Application Publication
(10) Pub. No.: US 2003/0017180 A1
(43) Pub. Date: Jan. 23, 2003

(54) Skin treatment compositions

(75) Inventors: David Acher, Beverley (GB); Severine Delaplace, Voiron (FR)

Correspondence Address: AKIN, GUMP, STRAUSS, HAUER & FELD, L.L.P., ONE COMMERCE SQUARE, SUITE 2200, 2005 MARKET STREET, PHILADELPHIA, PA, 19103, US

(73) Assignee: RECKITT BENCKISER (UK) LIMITED

(21) Appl. No.: 10/104,217

(22) Filed: Mar. 22, 2002

Related U.S. Application Data
(63) Continuation of application No. PCT/GB00/03632, filed on Sep. 22, 2000.

(30) Foreign Application Priority Data
Sep. 24, 1999 (EP) 99402344.8
Nov. 6, 1999 (GB) 9926243.8

(51) Int. Cl. A61K 35/78; A61K 7/00

(52) U.S. Cl. 424/401; 424/757; 424/764; 424/769

(57) ABSTRACT

A skin treatment composition is provided for the removal of wax residues from the skin after epilation of the skin. The composition comprises an internal aqueous phase, an external non-aqueous phase containing at least one coolant compound, and one or more surfactant(s).

### CROSS-REFERENCE TO RELATED APPLICATIONS

This application is a continuation of International Application No. PCT/GB00/03632, filed Sep. 22, 2000, which was published in the English language on Mar. 29, 2001, under International Publication No. WO 01/21146 A1, the disclosure of which is incorporated herein by reference.

### BACKGROUND OF THE INVENTION

The present invention is directed to compositions for the treatment of the skin and, in particular, to compositions for the removal of residues remaining after epilation of the skin.

Epilation is the technique whereby hair is removed from the skin by pulling the hair from its roots. Typically, a hot wax composition is applied to the area to be treated. Cotton strips are the applied over the hot wax composition and left in place for a short time, while the wax cools. The strips are then pulled off the skin, simultaneously removing the hair which adheres to the wax. Sugar-based compositions are also used in a similar way. Cold wax strips may be applied directly to the skin and then pulled off, removing the hair which adheres to the wax.

After epilation, residues of the epilation compositions remain on the skin. Furthermore, the skin may be reddened and feel sore from the epilation technique. Accordingly, it would be advantageous to provide a composition for the treatment of the skin after epilation, which can combine an oil for the removal of wax or other residues, with constituents which will provide a cooling and refreshing effect to the skin. Compounds which have a cooling effect, such as menthol and menthol derivatives, are active only in the presence of water, but not in the presence of oil.

International Patent Publication No. WO 98/15254 discloses cosmetic or dermatological micro-emulsion based gels which comprise a mixture of components comprising an aqueous phase, an oil phase, and one or more particular emulsifiers having an HLB value of from 2 to 14. The compositions are suggested for uses such as deodorants, make-up removal compositions, hair lotions, shower lotions and after shave lotions.

International Patent Publication No. WO 95/03772 discloses hydro-alcoholic micro-emulsions which include water, a C₁-C₄ alkanol and an oil which is a skin-nutrative oil, such as a vitamin oil. The compositions may be provided as lotions, sticks, roll-on formulations, mousses, aerosol sprays, etc.

International Patent Publication No. WO 96/41610 discloses a rinse-off cleansing composition, which comprises a first emulsion having a continuous phase comprising a surfactant and an internal phase comprising a second emulsion. The second emulsion has a continuous phase comprising a carrier, in which a cosmetic benefit agent is substantially insoluble, and an internal phase comprising the cosmetic benefit agent and an emulsifier.

International Patent Publication No. WO 93/07856 discloses a skin care composition in the form of a low pH aqueous gel. The compositions are stated to provide improved skin feel and residue characteristics. The gel comprises a non-ionic polyacrylaminde, a humectant, and an emollient and optionally a pharmaceutically or cosmetically active compound.

None of the compositions disclosed in these publications is a skin treatment composition for use after epilation of the skin.

### BRIEF SUMMARY OF THE INVENTION

Accordingly, the present invention provides a skin treatment composition for the removal of wax residues from the skin after epilation of the skin, which composition comprises an internal aqueous phase, an external non-aqueous phase containing at least one coolant compound and one or more surfactant(s). In a preferred embodiment, we have now developed a composition, which is an inverse micro-emulsion, which enables coolants and oil to be delivered to the skin in the same treatment composition.

### DETAILED DESCRIPTION OF THE INVENTION

The external non-aqueous phase is an oil, which may be non-polar or weakly polar. Preferably, the oil is one which is volatile enough for it to evaporate on exposure to air and therefore not remain on the surface to which the composition has been applied. For example, the oil may be a silicone oil, preferably one having surfactant properties (e.g., Abil k 520® from Goldschmidt AG or SF 1202 from GE Silicones), or a non-synthetic oil, such as olive, sunflower or jojoba oils or the like, or a hydrocarbon, such as C₁₁-C₁₂ paraffin or iso-paraffin oil (e.g., Isopar H from Exxon Chemical Co.).

The external, non-aqueous phase may include an antioxidant, such as vitamin E (for example, dl-alpha tocopherol from BASF), to reduce the tendency of some oils or active components to oxidize when exposed to atmospheric air on storage.

The coolant compound, which is incorporated into the oily phase, is preferably menthol or a menthol derivative, such as Questice L® from Quest, Frescolat® ML or MGA from Harmann & Reimer, or WS3 and WS23 from Chirex Ltd. The composition preferably also comprises ethanol, which boosts the refreshing effect of the coolant compound without disturbing the micro-emulsion system. The inverse micro-emulsion system of the present invention thus enables a single composition to deliver an oil to the skin for the removal of wax residues, at the same time as enabling coolant compounds to be delivered to the skin. The water in the aqueous phase diffuses onto the skin and enhances the action of the coolant compound.

In a particularly preferred embodiment of the invention, the composition is an inverse micro-emulsion, wherein the aqueous phase is a discontinuous phase in the form of aqueous droplets of nanometric diameter which are dispersed in a continuous non-aqueous phase. The aqueous droplets typically have a size of about 1 to 100 nm, preferably about 10 to 20 nm, more preferably around 5 nm, as measured in the non-aqueous phase by photon correlation spectroscopy.

In other embodiments, when not in the form of droplets, the composition exhibits various geometries of liquid crystals: hexagonal, cubic, sponge phase or lamellar, which collectively are referred to as structural micro-domains (or, more strictly, inverse micro-domains, since the micro-domains comprise water and not oil). Some of these compositions are thermotropic, because they reflect a narrow band of luminous frequency and thus exhibit an iridescent effect with variation of the temperature. In these embodiments, the internal aqueous phase is a continuous phase dispersed in a continuous non-aqueous phase.

Both spherical and micro-domain embodiments provide a transparent material that offers protection against oxidation to fragile components of the internal phase. In addition, the micro-domains have an aesthetically appealing visual effect.

Such compositions are preferably in the form of viscous liquids, solutions or gels, which are generally lipophilic and capable of dissolving lipophilic agents, such as liposoluble vitamins. Preferably, the compositions of the present invention are in the form of solutions (as in the case of droplet formation) or viscous gels (as in the case of micro-domain formation), more preferably solutions.

An advantage of the compositions of the present invention is that preservatives are not required, which avoids irritation of the skin. This results from the aqueous phase being protected from bacterial infection, because the ultra-small droplets (about 1 to about 100 nm) do not allow the development of bacteria therein, since the bacteria are too large, generally having a size of about 1 mm. The protection is further enhanced by ethanol. Additionally, the active component or components present in the aqueous phase are shielded from atmospheric air by the external non-aqueous phase and by any antioxidant present in this phase.

In addition to including an aqueous phase and a non-aqueous phase, the composition also includes one or more surfactants to facilitate the formation of a water-in-oil (as opposed to oil-in-water) emulsion.

The nature of the surfactant influences the phase structure of the composition. The phase structure is dependent on the packing parameter of the surfactant, as discussed in Intermolecular and Surface Forces by Jacob N. Israelachvili, page 380 et seq., Academic Press, Second Edition (1992) (incorporated herein by reference in its entirety), which is defined as:

V/AL

where V is the volume (solid angle) of the hydrophobic portion of the surfactant, A is the area of the hydrophilic portion of the surfactant, and L is the length of the hydrophilic portion. If the packing parameter has a value of about 0.9 to about 1.2, the composition exhibits a liquid crystal phase. If the packing parameter has a value of greater than 1.0, the composition is in the form of an inverse micro-emulsion.

The surfactant preferably has an HLB (hydrophile-lipophile balance), as discussed in Encyclopaedia of Emulsion Technology, edited by Paul Becher, pages 217-20, volume 1, Marcel Dekker (1983), of about 5 to about 16. An HLB value of about 8 to about 13 results in a composition of the liquid crystal phase type. An HLB value of about 10 to about 16 results in a composition of the micro-domain type. An HLB value of about 5 to about 10, advantageously about 6, results in a composition of the inverse micro-emulsion type. The relationship between the packing parameter and the HLB is reported in Proceedings of the First World Congress on Emulsions, by Israelachvili, page 58 et seq, Paris (1993).

A suitable surfactant is that known under the designation LRI®, which is a mixture of PPG 26 Buteth 26 and hydrogenated caster oil available from Les Colorants Wackkers. Other suitable surfactants are PEG 30 dipolyhydroxystearate and polyoxypropylene 15 stearyl ether (Arlacel P135® and Arlamol E® from ICI). Where the non-aqueous phase is a silicone oil, then preferably a silicone-based surfactant is chosen. The surfactant(s) may be or include one or more polymeric surfactant(s), for example further to facilitate formation of a water-in-oil (as opposed to oil-in-water) composition.

Optionally, therefore, the compositions according to the invention may further comprise a polymer that may also affect the phase structure of the composition. Preferably, the polymer is one that enables the interface between the water and oil phases to become more stable and rigid than in the absence of the polymer. Accordingly, the packing parameter of the polymer may be defined in a way corresponding to that, above, for the surfactant.

Suitable polymers are those having a hydrophobic portion. Suitable polymers are non-ionic polyoxyethlenes, such as those of the Elfacos® series from Akzo. These are polyethylene glycol dodecyl glycols, and methoxy PEG 22 dodecyl glycol (Elfacos E200®) is particularly preferred. Other suitable polymers are PEG 22 dodecyl glycol copolymer and PEG 45 dodecyl glycol copolymer. Alternatively, PEG 30 (dipolyhdroxy stearate, available from ICI, UK as Arlacel P135®) may be used.

In particular, in the case where the polymer does not have a hydrophobic portion, a co-surfactant may be advantageously incorporated, in particular to assist in the geometry of the interface so that an inverse emulsion, rather than a regular emulsion, is formed. Typical co-surfactants are C₃-C₁₂ alcohols, such as pentanol, octanol, dodecanol, isopropanol, ethoxydiglycol, or Arlamol E® (PPG-15 stearyl ether from ICI). The use of longer chain alcohols as co-surfactants is preferred, since they are less likely to irritate the skin. Particularly preferred is dodecanol-1, available from Condea under the trademark Nacol 1299®.

The HLB values of the co-surfactants and other ingredients should be such as to enable the overall HLB of the composition to fall within the ranges specified above in relation to the surfactant.

The compositions of the present invention may contain, in addition to a coolant, other active components which have an effect on the skin following epilation. Examples of suitable additional components are actives and plant extracts which are hydrosoluble or liposoluble, such as: actives for blood circulation problems (in particular when using hot waxes), such as vaso-protectors, vaso-constrictors, veinous toning agents; cell turnover stimulants or cell healing compounds; anti-inflammatories; disinfectants or anti-bacterials; soothing components or moisturisers.

The compositions of the invention are preferably in the form of a homogenous phase lotion or a transparent, viscous gel. In the case where it is a lotion, the system is a micro-emulsion, and the surfactant and the co-surfactant surround very small droplets of water (of preferably nanometric diameter), and the non-aqueous phase (oily phase) contains the coolant compound, ethanol and the lipophilic antioxidant. In the case where the composition is a gel, the system includes liquid crystal domains, and the surfactant and co-surfactant are arranged so as to form a cubic phase, hexagonal phase or lamellar phase.

In the case where the composition of the present invention is in the form of an inverse micro-emulsion, the water that enables the action of the skin coolants is in the form of very small domains (micrometric or, preferably, nanometric domains). These are able to penetrate the skin much more readily than in conventional water-in-oil compositions (conventional inverse emulsions), and hence the skin treatment efficiency is improved.

Typical ingredients and concentrations of a skin treatment composition in accordance with the present invention are as shown in the following table:

| Ingredients | % w/w composition |
|---|---|
| Aqueous Phase | |
| Water | 0.1 to 20 |
| Ethanol | 0 to 20 |
| Non-aqueous Phase | |
| Isopar H and/or silicone oil | 20 to 60 |
| Menthol | 0.05 to 0.5 |
| Cooling agents | 0.1 to 3 |
| Anti-oxidant | <0.5 |
| Interface | |
| Surfactant | 10 to 40 |
| Co-surfactant | 5 to 20 |

The present invention will be further described with reference to the following specific, non-limiting examples:

### EXAMPLE 1

| Phase | Ingredient | % w/w Composition |
|---|---|---|
| Non-aqueous phase | Isopar H (Exxon) | 58.7 |
| | Anti-oxidant | 0.1 |
| | Dye | q.s. |
| | Perfume | q.s. |
| | Frescolat ML ® (H&R) | 1.0 |
| | L-Menthol | 0.2 |
| Surfactant | Elfacos 200 ® (Akzo Nobel) | 17.5 |
| Co-Surfactant | Dodecanol-1 | 12.25 |
| Aqueous Phase | Water | 5 |
| | Ethanol | 5.25 |

First, the aqueous phase ingredients were combined. Then, the surfactant and co-surfactant were combined with the non-aqueous phase, and the two phases were mixed at room temperature. An inverse water-in-oil micro-emulsion was formed.

### EXAMPLE 2

An inverse water-in-oil micro-emulsion was prepared according to the method of Example 1 from the following ingredients:

| | | % w/w |
|---|---|---|
| Phase | Ingredient | Composition |
| Non-aqueous phase | Isopar H (Exxon) | 27.8 |
| | Dye | q.s. |
| | Perfume | q.s. |
| | Questice L ® (Quest) | 2.0 |
| | L-Menthol | 0.2 |
| Surfactant | LRI ® (Les Colorants Wackkers) | 25 |
| Co-Surfactant | Octanol | 17.5 |
| Aqueous Phase | Water | 20 |
| | Ethanol | 7.5 |

### EXAMPLE 3

A transparent gel was prepared from the following ingredients:

| | | % w/w |
|---|---|---|
| Phase | Ingredient | Composition |
| Non-aqueous phase | Isopar H (Exxon) | 39.52 |
| | Frescolat ML ® (H&R) | 1.0 |
| | Menthol | 0.2 |
| Surfactant | LRI ® (Les Colorants Wackkers) | 26.34 |
| Co-Surfactant | Dodecanol-1 | 13.18 |
| Aqueous Phase | Water | 19.76 |

This transparent gel contains inverse micro-domains which comprise water.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A skin treatment composition for removal of wax residues from skin after epilation of the skin, the composition comprising an internal aqueous phase, an external non-aqueous phase containing at least one coolant compound, and at least one surfactant.

2. The composition as claimed in claim 1, wherein the external non-aqueous phase comprises a non-polar or weakly polar oil.

3. The composition as claimed in claim 2, wherein the oil is selected from the group consisting of silicone oils, non-synthetic oils, and hydrocarbons.

4. The composition as claimed in claim 3, wherein the oil is a non-synthetic oil selected from the group consisting of olive, sunflower and jojoba oils.

5. The composition as claimed in claim 3, wherein the oil is a hydrocarbon selected from the group consisting of C₁₁ -C₁₂ paraffin and iso-paraffin oils

6. The composition as claimed in claim 1, wherein the coolant compound comprises menthol or a menthol derivative.

7. The composition as claimed in claim 1, wherein the aqueous phase further comprises ethanol.

8. The composition as claimed in claim 1, wherein the composition is an inverse micro-emulsion in which the aqueous phase is a discontinuous phase in a form of aqueous droplets of nanometric diameter, as measured by photon correlation spectroscopy, the droplets being dispersed in the non-aqueous phase which is continuous.

9. The composition according to claim 8, wherein the aqueous droplets have a diameter of about 1 to about 100 nm, as measured by photon correlation spectroscopy.

10. The composition as claimed in claim 1, wherein the composition is an inverse micro-emulsion in which the aqueous phase is a discontinuous phase in a form of structural micro-domains which are dispersed in the non-aqueous phase which is continuous.

11. The composition as claimed in claim 1, further comprising at least one of a polymer having a hydrophobic portion and a co-surfactant, where the surfactant/polymer/co-surfactant system has an HLB in a range of about 5 to about 16.

12. The composition as claimed in claim 1, the aqueous or non-aqueous phase further comprises at least one active component which has an effect on the skin.
